(19) United States
(12) Patent Application Publication
(10) Pub. No.: US 2019/0168025 A1
(43) Pub. Date: Jun. 6, 2019

(54) IMAGE-GUIDED RADIATION THERAPY

(71) Applicant: Varian Medical Systems International AG.

(72) Inventors: Timo KOPONEN, Helsinki (FI); Hanna KOIVUNORO, Helsinki (FI)

(21) Appl. No.: 15/833,919

(22) Filed: Dec. 6, 2017

(51) Int. Cl. A61N 5/10

(52) U.S. Cl. CPC A61N 5/107; A61N 5/1049; A61N 2005/1061; A61N 5/1045; A61N 5/1047; A61N 5/1067

(57) ABSTRACT

A system includes a first source of a radiation treatment beam, a first imager and a second imager, a first imaging beam source that directs a first imaging beam through a target and to the first imager to produce first images, a second imaging beam source that directs a second imaging beam through the target and to the second imager to produce second images, and a controller coupled to the first source, to the first and second imaging beam sources, and to the first and second imagers. The controller uses the first images and the second images to detect a change in position of the target and, in response to the change, adjusts the radiation treatment beam and/or the position of the target to compensate for the change while the radiation treatment beam is on.

### BACKGROUND

The use of radiation therapy to treat cancer is well known. Typically, radiation therapy involves directing a radiation treatment beam of high energy proton, photon, ion, or electron radiation (“therapeutic radiation”) into a target or target volume (e.g., a tumor or lesion).

Before a patient is treated with radiation, a treatment plan specific to that patient is developed. The plan defines various aspects of the therapy using simulations and optimizations based on past experiences. In general, the purpose of the treatment plan is to deliver sufficient radiation to the target while minimizing exposure of surrounding normal, healthy tissue to the radiation.

A patient typically first receives a CT (computed tomography) scan used to simulate the patient's treatment. A simulated treatment plan defines beam orientations and corresponding particle fluences to generate a 3D (three-dimensional) dose distribution that best achieves the physician's prescription and/or intent. Once the treatment plan has been defined, treatment can commence.

Differences in the patient's physiology at the time of delivery of each treatment fraction (each treatment session), compared to the CT simulation from which the treatment plan was derived, result in some degree of uncertainty in the actual treatment. One way to address this uncertainty is to define large margins in the treatment plan, but this increases the risk of side effects.

Motion of the target because of patient movement, respiratory motion (breathing), cardiac motion (heart function), and the like can further compound the treatment uncertainty. If these types of motion are not taken into consideration during treatment, the radiation treatment beam may miss the target (and hit healthy tissue) or the beam's radiation may not be properly distributed across and into the target.

Various techniques are currently employed to manage motion during treatment in order to minimize the difference between the planned and delivered dose to the patient, such as by having the patient hold their breath or by gating the treatment beam. Each of these techniques has associated benefits and but also associated drawbacks.

For example, many patients do not have the ability to hold their breath for more than a few seconds and so they cannot hold their breath for the duration of the treatment fraction. Thus, treatment has to be frequently halted and then restarted. Consequently, the length of each treatment session is considerably extended, which can distress and inconvenience the patient. Gating, in which the radiation treatment beam is turned off whenever the target moves outside of the beam, also has this disadvantage.

Contemporary modulated radiotherapy techniques like intensity-modulated radiation therapy (IMRT) and intensity-modulated proton therapy (IMPT) allow improved dose coverage of targets with lower doses outside the target, but because of patient motion, healthy tissue is still often unavoidably irradiated with low doses that can increase the risk of secondary cancer.

### SUMMARY

Embodiments according to the invention provide systems and methods for treating a moving target in three dimensions. In embodiments, the target is imaged in real time while the radiation treatment beam is on. If the position of the target changes, then the radiation treatment beam is changed to compensate for the change in real time, while the beam is on and irradiating the target.

In embodiments, a system includes a source of a radiation treatment beam, a first imager and a second imager, a first imaging beam source that directs a first imaging beam through a target and to the first imager to produce first images, a second imaging beam source that directs a second imaging beam through the target and to the second imager to produce second images, and a controller. In an embodiment, the controller uses the first images and the second images to detect a change in position of the target and, in response to the change, the controller adjusts the radiation treatment beam and/or the position of the target to compensate for the change while the beam is on.

For example, if the target moves laterally during treatment (in a direction orthogonal to the direction of the radiation treatment beam), then the beam direction and/or angle can be changed, also in real time while the beam is on, to track the motion of the target so that the beam continues to intersect the target. Alternatively, if the target moves laterally during treatment, then the target can be further moved (e.g., by moving the patient support chair, couch, or table) in real time while the radiation treatment beam is on so that the target continues to be aligned with the path of the beam. As another alternative, both the radiation treatment beam and the target can be moved so that the beam continues to intersect the target. In some embodiments, if the position of the target changes axially (along or roughly parallel to the axis of the radiation treatment beam), then the range of the beam can be changed (adjusted or shifted) so that the beam continues to intersect the target.

In embodiments, filters are placed between each of the imaging beam sources and the respective imager. Each filter can include different regions of different filter materials. Generally speaking, the filters can improve the quality of the images acquired by the imagers, making the target more visible in the images.

In embodiments, the first imaging beam is produced at a first voltage and the second imaging beam is produced at a second, different voltage. That is, the first and second imaging beams have different energies. The voltages or energies can be chosen based on, for example, the target's composition and density, to make the target more visible in the images.

In embodiments, the first imaging beam and the second imaging beam are each produced alternately at a first voltage and at a second, different voltage. That is, the first and second imaging beams alternate between different energies. Alternating voltages used to produce the imaging beams or alternating the beam energies can improve soft tissue contrast, to make the target more visible in the images.

Thus, in general, embodiments according to the invention are able to localize the target and surrounding healthy tissue. Target motion is imaged in real time during treatment while the radiation treatment beam is on and is irradiating the target. Based on the acquired images, the radiation treatment beam and/or the position of the target and/or range of the beam can be interactively guided in real time (while the beam is on and is irradiating the target) to provide a desired or optimal dose distribution into and across the target while sparing the surrounding healthy tissue.

Consequently, breath control is not required, making it more convenient for the patient and reducing the possibility of mistreatment due to improper breath control. Gating of the radiation treatment beam is also not required, thereby reducing treatment time and making treatment more convenient for the patient. Because the location of the target is known in real time, margins in the treatment plan (e.g., the planned irradiation volume) can be reduced without risk of compromising target dose coverage, lowering the risk of associated side effects.

In addition to intensity-modulated radiation therapy (IMRT) and intensity-modulated proton therapy (IMPT), embodiments according to the invention can be used in spatially fractionated radiation therapy including high-dose spatially fractionated grid radiation therapy and microbeam radiation therapy.

These and other objects and advantages of embodiments according to the present invention will be recognized by one skilled in the art after having read the following detailed description, which are illustrated in the various drawing figures.

This summary is provided to introduce a selection of concepts in a simplified form that is further described below in the detailed description that follows. This summary is not intended to identify key features or essential features of the claimed subject matter, nor is it intended to be used to limit the scope of the claimed subject matter.

### BRIEF DESCRIPTION OF DRAWINGS

The accompanying drawings, which are incorporated in and form a part of this specification and in which like numerals depict like elements, illustrate embodiments of the present disclosure and, together with the detailed description, serve to explain the principles of the disclosure.

FIG. 1 is a block diagram of an example of a computing system upon which the embodiments described herein may be implemented.

FIG. 2 is a block diagram showing selected components of a radiation therapy system upon which embodiments according to the present invention can be implemented.

FIG. 3A illustrates selected components of the radiation therapy system upon which embodiments according to the present invention can be implemented.

FIGS. 3B and 3C illustrate filters in the radiation therapy system in embodiments according to the invention.

FIGS. 4A, 4B, 4C, and 4D are examples of arrangements of different split beam filters in embodiments according to the invention.

FIG. 5 is an example illustrating the use of an imaging system to detect motion of a target and to acquire positional information that can be used to locate the target in embodiments according to the present invention.

FIG. 6 is a flowchart of an example of computer-implemented operations for image-guided radiation therapy in embodiments according to the present invention.

FIG. 7 is a flowchart of an example of computer-implemented operations for image-guided radiation therapy in embodiments according to the present invention.

### DETAILED DESCRIPTION

Reference will now be made in detail to the various embodiments of the present disclosure, examples of which are illustrated in the accompanying drawings. While described in conjunction with these embodiments, it will be understood that they are not intended to limit the disclosure to these embodiments. On the contrary, the disclosure is intended to cover alternatives, modifications and equivalents, which may be included within the spirit and scope of the disclosure as defined by the appended claims. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

Some portions of the detailed descriptions that follow are presented in terms of procedures, logic blocks, processing, and other symbolic representations of operations on data bits within a computer memory. These descriptions and representations are the means used by those skilled in the data processing arts to most effectively convey the substance of their work to others skilled in the art. In the present application, a procedure, logic block, process, or the like, is conceived to be a self-consistent sequence of steps or instructions leading to a desired result. The steps are those utilizing physical manipulations of physical quantities. Usually, although not necessarily, these quantities take the form of electrical or magnetic signals capable of being stored, transferred, combined, compared, and otherwise manipulated in a computing system. It has proven convenient at times, principally for reasons of common usage, to refer to these signals as transactions, bits, values, elements, symbols, characters, samples, pixels, or the like.

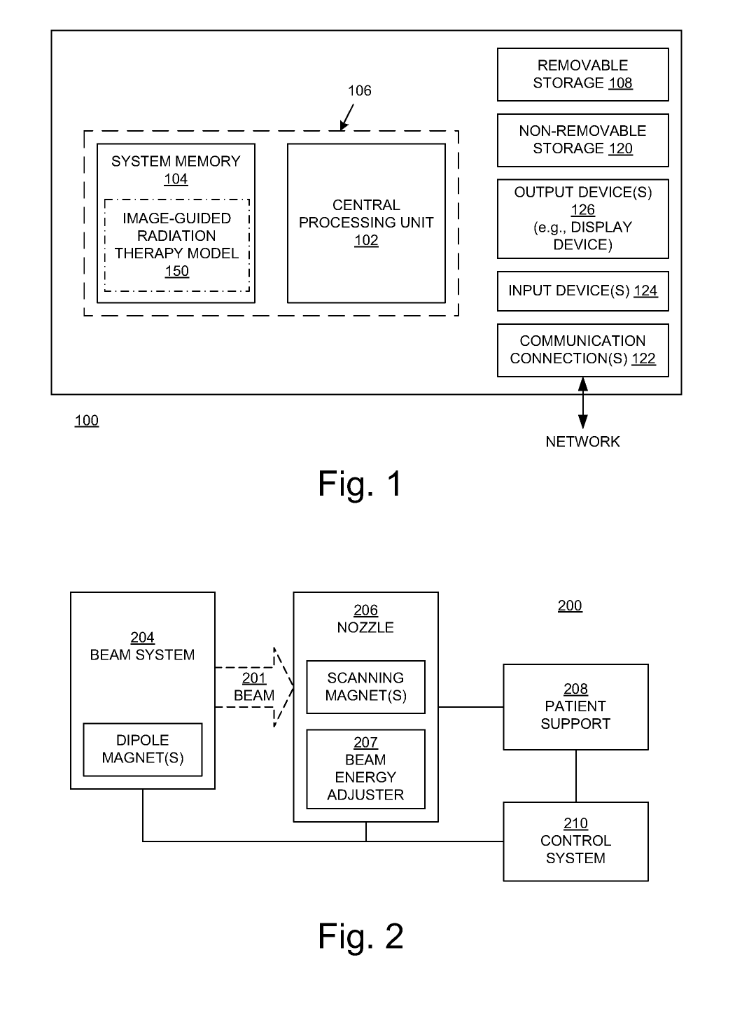

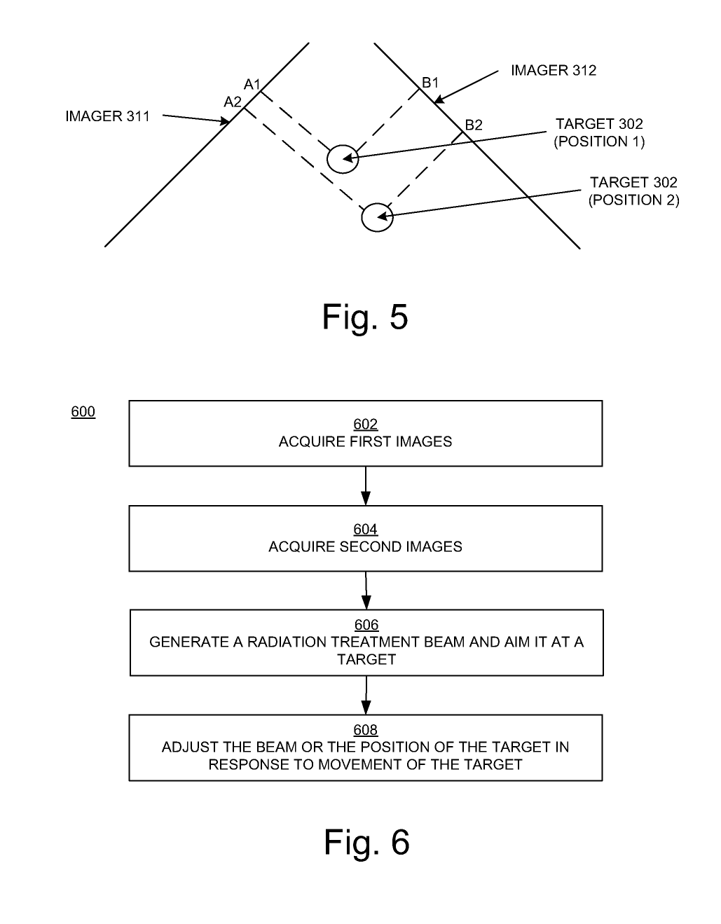

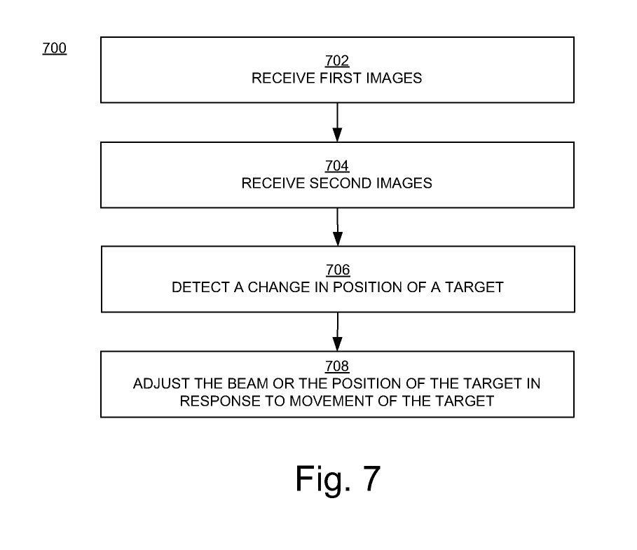

It should be borne in mind, however, that all of these and similar terms are to be associated with the appropriate physical quantities and are merely convenient labels applied to these quantities. Unless specifically stated otherwise as apparent from the following discussions, it is appreciated that throughout the present disclosure, discussions utilizing terms such as “determining,” “accessing,” “directing,” “controlling,” “receiving,” “changing,” “detecting,” “adjusting,” or the like, refer to actions and processes (e.g., the flowcharts of FIGS. 6 and 7) of a computing system or similar electronic computing device or processor (e.g., the computing system 100 of FIG. 1). The computing system or similar electronic computing device manipulates and transforms data represented as physical (electronic) quantities within the computing system memories, registers or other such information storage, transmission or display devices. Terms such as “voltage” or “energy” generally mean an amount of voltage or energy; the use of such terms will be clear from the context of the surrounding discussion.

Portions of the detailed description that follows are presented and discussed in terms of a method. Although steps and sequencing thereof are disclosed in figures herein (e.g., FIGS. 6 and 7) describing the operations of this method, such steps and sequencing are exemplary. Embodiments are well suited to performing various other steps or variations of the steps recited in the flowchart of the figure herein, and in a sequence other than that depicted and described herein.

Embodiments described herein may be discussed in the general context of computer-executable instructions residing on some form of computer-readable storage medium, such as program modules, executed by one or more computers or other devices. By way of example, and not limitation, computer-readable storage media may comprise non-transitory computer storage media and communication media. Generally, program modules include routines, programs, objects, components, data structures, etc., that perform particular tasks or implement particular abstract data types. The functionality of the program modules may be combined or distributed as desired in various embodiments.

Computer storage media includes volatile and nonvolatile, removable and non-removable media implemented in any method or technology for storage of information such as computer-readable instructions, data structures, program modules or other data. Computer storage media includes, but is not limited to, random access memory (RAM), read only memory (ROM), electrically erasable programmable ROM (EEPROM), flash memory or other memory technology, compact disk ROM (CD-ROM), digital versatile disks (DVDs) or other optical storage, magnetic cassettes, magnetic tape, magnetic disk storage or other magnetic storage devices, or any other medium that can be used to store the desired information and that can be accessed to retrieve that information.

Communication media can embody computer-executable instructions, data structures, and program modules, and includes any information delivery media. By way of example, and not limitation, communication media includes wired media such as a wired network or direct-wired connection, and wireless media such as acoustic, radio frequency (RF), infrared and other wireless media. Combinations of any of the above can also be included within the scope of computer-readable media.

FIG. 1 shows a block diagram of an example of a computing system 100 upon which the embodiments described herein may be implemented. In its most basic configuration, the system 100 includes at least one processing unit 102 and memory 104. This most basic configuration is illustrated in FIG. 1 by dashed line 106. The system 100 may also have additional features and/or functionality. For example, the system 100 may also include additional storage (removable and/or non-removable) including, but not limited to, magnetic or optical disks or tape. Such additional storage is illustrated in FIG. 1 by removable storage 108 and non-removable storage 120. The system 100 may also contain communications connection(s) 122 that allow the device to communicate with other devices, e.g., in a networked environment using logical connections to one or more remote computers.

The system 100 may also include input device(s) 124 such as keyboard, mouse, pen, voice input device, touch input device, etc. Output device(s) 126 such as a display device, speakers, printer, etc., may also be included.

In the example of FIG. 1, the memory 104 includes computer-readable instructions, data structures, program modules, and the like associated with an image-guided radiation therapy model 150. However, the image-guided radiation therapy model 150 may instead reside in any one of the computer storage media used by the system 100, or it may be distributed over some combination of the computer storage media, or it may be distributed over some combination of networked computers. Operations performed or directed by the image-guided radiation therapy model 150 are described below.

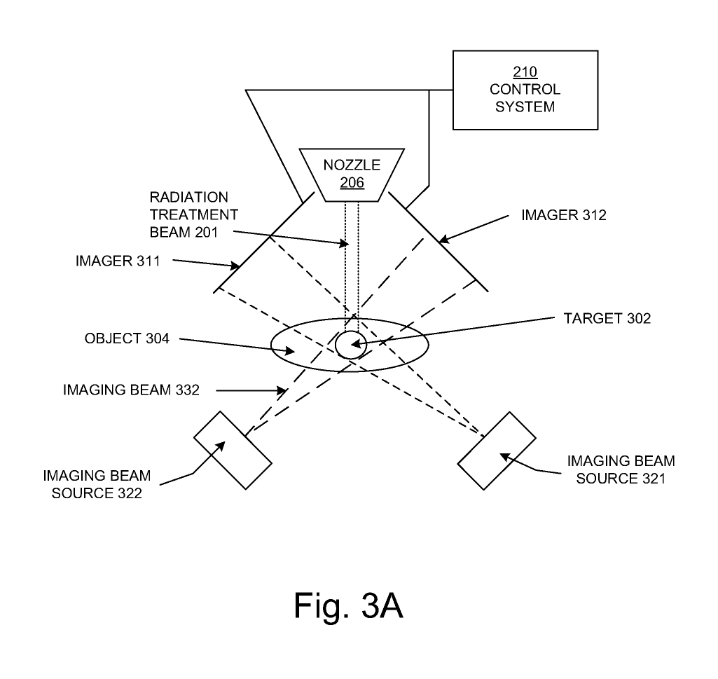

FIG. 2 is a block diagram showing selected components of a radiation therapy system 200 upon which embodiments according to the present invention can be implemented. In the example of FIG. 2, the radiation therapy system 200 includes a beam system 204, a nozzle 206, a patient support device 208, and a control system 210.

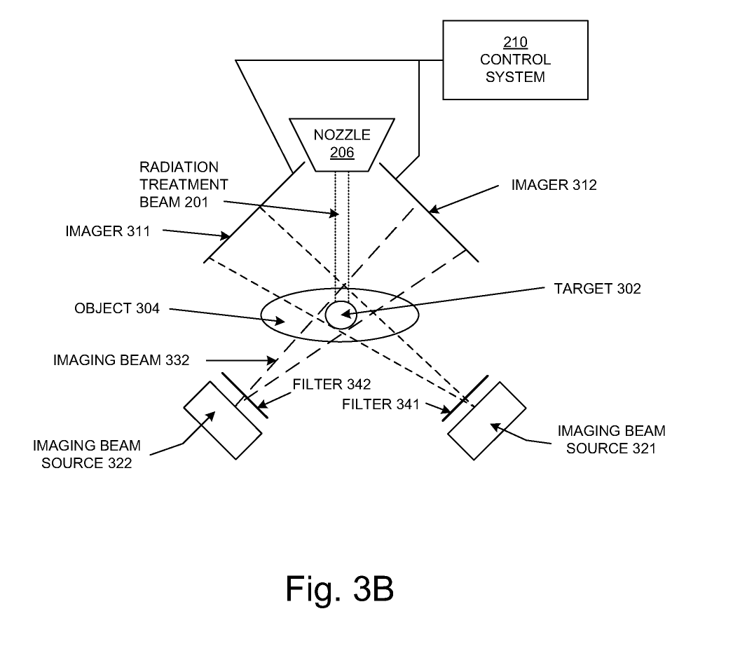

The control system 210 of FIG. 2, which may also be referred to herein simply as a controller, receives and implements a prescribed treatment plan and also implements the image-guided radiation therapy model 150 (FIG. 1). In embodiments, the control system 210 includes one or more computer systems (e.g., like the computing system 100 of FIG. 1). The control system 210 can receive data regarding operation of the radiation therapy system 200. Specifically, as will be described further below, the control system 210 can receive information from an imaging system (see FIG. 3A). The control system 210 can control parameters of the beam system 204, nozzle 206, and patient support device 208, including parameters such as the energy, intensity, direction, size, range, and/or shape of the radiation treatment beam 201, the orientation of the nozzle, and the position of the patient support device, according to information and data it receives from the imaging system and according to the prescribed treatment plan.

The beam system 204 generates and transports a radiation treatment beam 201 to the nozzle 206. In general, the radiation treatment beam 201 can be a proton beam, electron beam, photon beam, ion beam, or atom nuclei beam (e.g., carbon, helium, and lithium).

In embodiments, depending on the type of beam, the beam system 204 includes components (e.g., dipole magnets, also known as bending magnets) that direct (e.g., bend, steer, or guide) the radiation treatment beam 201 through the system in a direction toward and into the nozzle 206. In embodiments, the radiation treatment system 200 may also include one or more multileaf collimators (MLCs); each MLC leaf can be independently moved back-and-forth by the control system 210 to dynamically shape an aperture through which the radiation treatment 201 beam can pass, to block or not block portions of the beam and thereby control beam shape and exposure time. The beam system 204 may also include components that are used to adjust (e.g., reduce) the energy of the radiation treatment beam 201 entering the nozzle 206.

The nozzle 206 may be mounted on or be a part of a gantry that can be moved relative to the patient support device 208, which may also be moveable. In embodiments, the beam system 204 is also mounted on or is a part of the gantry. In another embodiment, the beam system 204 is separate from (but in communication with) the gantry.

The nozzle 206 includes components (e.g., scanning magnets) used to direct (aim) the radiation treatment beam 201 toward various locations (a target) within an object (e.g., a patient) supported on the patient support device 208 in a treatment room. In embodiments, the patient support device 208 is a table or couch that supports the patient in a supine or prone position. In another embodiment, the patient support device 208 is a moveable chair in which the patient sits.

A target may be, for example, an organ, a portion of an organ (e.g., a volume or region within the organ), a tumor, or diseased tissue.

As noted above, the radiation treatment beam 201 entering the nozzle 206 has a specified energy. Thus, in some embodiments, the nozzle 206 includes one or more components that affect (e.g., decrease, modulate) the energy of the radiation treatment beam 201. The beam energy adjuster 207 is a component or components that affect the energy of the particles in the radiation treatment beam 201, in order to control the range of the beam (e.g., the extent that the beam penetrates into a target), to control the dose delivered by the beam, and/or to control the depth dose curve of the beam, depending on the type of beam. For example, for a proton beam or an ion beam that has a Bragg peak, the beam energy adjuster 207 can control the location of the Bragg peak in the target. In various embodiments, the beam energy adjuster 207 includes a range modulator, a range shifter, or both a range modulator and a range shifter.

FIG. 3A illustrates selected components of the radiation therapy system 200 upon which embodiments according to the present invention can be implemented. As mentioned above, the nozzle 206 is used to direct the radiation treatment beam 201 toward and into a target 302 within an object 304 (e.g., a patient) supported on the patient support device 208 (not shown in FIG. 3A).

In embodiments, the radiation therapy system 200 includes a first imager 311, a second imager 312, a first imaging beam source 321, and a second imaging beam source 322 in addition to those already described. The first imaging beam source 321 can direct a first imaging beam 331 through the target 302 and to the first imager 311 to produce first images, and the second imaging beam source 322 can direct a second imaging beam 332 through the target and to the second imager 312 to produce second images. An imaging beam may also be known as an imaging field. In an embodiment, the first imaging beam 331 and the second imaging beam 332 are x-ray beams, and the first imaging beam source 321 and the second imaging beam source 322 are x-ray tubes. For ease of discussion, the imaging beam sources, the imagers, and the imaging beams may be collectively referred to herein as the imaging system. The radiation therapy system 200 may include more than two imaging beam sources, imaging beams, and imagers.

As will be described in greater detail below, the imaging system is used in real time, while the radiation treatment beam is turned on and is irradiating the target during treatment, to acquire images of the target 302 that can be used to detect and monitor motion of the target. The imaging system can also be used to detect and monitor motion of tissue surrounding the target 302.

The imagers 311 and 312 are arranged at different angles relative to, for example, the incident radiation treatment beam 201. In an embodiment, the imagers 311 and 312 are at an angle of 90 degrees relative to each other. The imagers 311 and 312 are in communication with the control system 210 of FIG. 2 so that they can send or transmit images (image data) to the control system. The control system 210, in turn, can use that information to determine the position of the target 302.

In embodiments, the first and second imaging beam sources 321 and 322 are multi-energy beam sources. That is, the first and second imaging beam sources 321 and 322 produce imaging beams 331 and 332 that have a range of energies. The range of energies may be different for each of the imaging beams 331 and 332, or they may be the same. For example, the first and second imaging beam sources 321 and 322 may each produce x-ray beams at (with a power source of) 80-140 kilovolts (kV). Such beams are commonly referred to as 80-140 kV beams.

In an embodiment, the imaging beams 331 and 332 are each produced with the same voltage. For example, the first and second imaging beam sources 321 and 322 each produce x-rays at (with a power source of) 100 kV. Such a beam is commonly referred to as a 100 kV beam.

In another embodiment, the first and second imaging beam sources 321 and 322 produce imaging beams 331 and 332 that have different energies. For example, the imaging beam 331 may be an 80 kV beam and the imaging beam 332 may be a 140 kV beam. In an embodiment, the energy for each of the imaging beams 331 and 332 is separately chosen to maximize the visibility of the target 302 acquired by the imaging system.

In another embodiment, the voltages of the first and second imaging beam sources 321 and 322 can be changed (alternated) to produce imaging beams 331 and 332 that alternate between two different energies. In an embodiment, the first and second imaging beam sources 321 and 322 are alternated between their respective minimum and maximum voltages. Thus, the imaging beams 331 and 332 each may alternate between their minimum and maximum energies (e.g., between an 80 kV beam and a 140 kV beam). In another embodiment, at least one of the energies for each of the imaging beams 331 and 332 is chosen to maximize the visibility of the target 302 acquired by the imaging system. The first and second imaging beam sources 321 and 322 can operate independently of one another, so they can pulse at different rates and/or so that one of the imaging beams is at its maximum energy while the other is at its minimum energy, and vice versa. By pulsing or alternating the energy of the imaging beams 331 and 332, soft tissue contrast is improved and target visibility is enhanced.

In embodiments, filters 341 and 342 are placed between each of the imaging beam sources 321 and 322 and the respective imager 311-312. Each filter can include different regions of different materials. As such, each filter may be referred to as a split filter.

Generally speaking, the filters can improve the quality of the images acquired by the imagers 311 and 312, making the target more visible in the images. The filters 342 and 342 can trim the spectrum of the imaging beams 331 and 332. For example, a filter can trim portions of an imaging beam that have energies greater than and less than the energy of a 140 kV beam, to more precisely achieve a 140 kV beam. One filter can be optimized for one beam energy level and the other filter can be optimized for another beam energy level.

In an embodiment, the filters 341 and 342 are placed directly in front of the imaging beam sources 321 and 322, respectively, as shown in FIG. 3B. In another embodiment, the filters 341 and 342 are placed directly in front of the imagers 311 and 312, respectively, as shown in FIG. 3C.

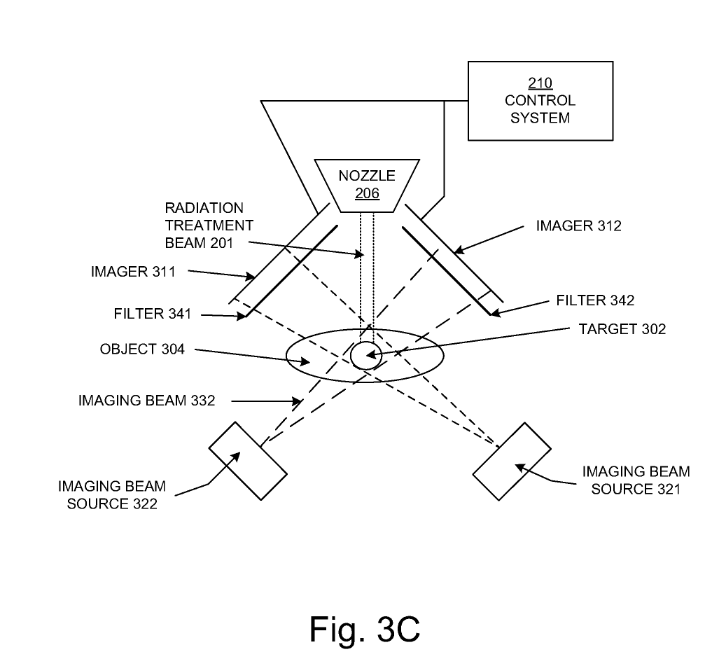

FIGS. 4A, 4B, 4C, and 4D are examples of arrangements of the split beam filters 341 and 342. The filters 341 and 342 may be arranged differently from one another. In each of these figures, the filter material of region 1 is different from the filter material of region 2. The filter materials may be different types of metal that maximize the signal contrast when the target 302 moves across the projected boundaries between regions 1 and 2. Other arrangements of filter materials, including more than two filter materials, may be used.

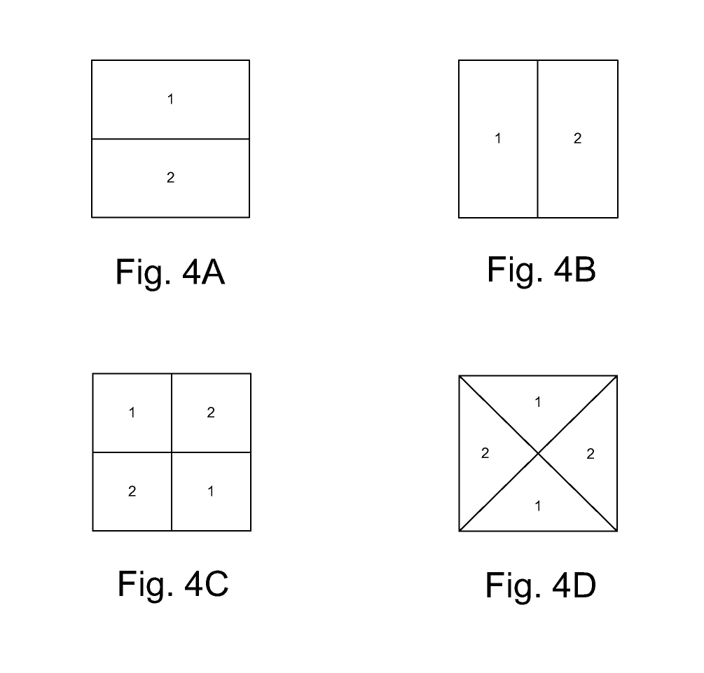

FIG. 5 is an example illustrating the use of the imaging system to detect motion of the target 302 and to acquire positional information that can be used to locate the target in embodiments according to the present invention. In the example of FIG. 5, the target 302 is initially at position 1, and the projected image of the target 302 is at position A1 on the imager 311 and at position B1 on the imager 312. Because of the different projection angles of the imagers 311 and 312, it is possible for the control system 210 (FIG. 2) to locate the target 302 by combining the images (image data) from the imagers 311 and 312. When the target 302 moves to position 2, the projected image of the target 302 is at position A2 on the imager 311 and at position B2 on the imager 312. Again, the control system 210 can locate the target 302 by combining the images (image data) from the imagers 311 and 312. Alternatively, the amount of displacement between the positions A1 and A2, and the amount of displacement between the positions B1 and B2, can be used to locate position 2 of the target 302.

Movement of the target 302 can be detected by comparing the images acquired when the target is at position 2 with the images acquired when the target is at position 1. It is not necessary for the control system 210 to continuously calculate the position of the target 302. Instead, the control system 210 can calculate the target's position in response to determining that the target has moved.

Images can continue to be acquired during the radiation treatment session. The frame rate at which images are acquired can be held constant or can be increased or decreased during the session.

For radiation treatment planning, the free breathing respiratory phases of the patient are imaged with computed tomography (CT) or with another methodology or technology that can provide that information. Treatment planning is performed for multiple respiratory phases, so that sufficient anatomical information is available for delivering the radiation treatment beam accurately during any phase of the breathing cycle, taking into account anatomical changes such as target and critical organ movement and deformation during treatment. In other words, as a result of treatment planning in advance of the treatment itself, there are a number of images generated that show the (expected) position of the target during treatment. Thus, as an alternative to or in addition to the above, movement of the target 302 can also be detected by comparing the images of target 302 at any time with those preexisting images. In response to detecting movement of the target 302, the new position of the target can be determined as described above.

If the target 302 moves laterally during treatment (in a direction orthogonal to the direction of the radiation treatment beam 201), then the direction or angle of the beam is changed using the scanning magnets in the nozzle 206 (FIG. 2) and/or by moving the nozzle or gantry, also in real time while the radiation treatment beam is on, to track the motion of the target so that the beam continues to intersect the target. Alternatively, if the target 302 moves laterally during treatment, then the target can be further moved (e.g., by moving the patient device 208 of FIG. 2) in real time while the radiation treatment beam 201 is on, so that the target continues to be aligned with the path of the beam. As another alternative, both the radiation treatment beam 201 and the target 302 can be moved so that the beam continues to intersect the target.

If the target 302 moves axially during treatment (along or roughly parallel to the axis of the radiation treatment beam 201), then the range of the beam can be changed (adjusted or shifted) using the beam energy adjuster 207 (FIG. 2) so that the beam continues to intersect the target. For example, for a proton beam or an ion beam that has a Bragg peak, the beam energy adjuster 207 can control the location of the Bragg peak so that it remains in the target 302.

More specifically, in embodiments, the radiation treatment beam 201 (not shown in FIG. 5) is first aimed at position 1. Based on the positional information obtained using the imaging system, when the target 302 moves laterally to position 2, the radiation treatment beam 201 is then aimed at position 2. Significantly, the aiming of the radiation treatment beam 201 from position 1 to position 2 is done in real time while the beam is turned on and is irradiating the target 302. That is, the radiation treatment beam 201 is not gated (it is not turned off when it is being aimed at position 2).

In other embodiments, the radiation treatment beam 201 (FIG. 2; not shown in FIG. 5) is first aimed at position 1. Based on the positional information obtained using the imaging system, when the target 302 moves laterally to position 2, the patient support device 208 is moved so that the radiation treatment beam 201 and the target remain aligned. Significantly, the movement of the patient support device 208 is done in real time while the beam is turned on and is irradiating the target 302. That is, the radiation treatment beam 201 is not gated.

In other embodiments, the radiation treatment beam 201 (FIG. 2; not shown in FIG. 5) is first aimed at position 1. Based on the positional information obtained using the imaging system, when the target 302 moves axially to position 2, the range of the radiation treatment beam 201 is adjusted using the beam energy adjuster 207. Significantly, the range of the radiation treatment beam 201 is adjusted in real time while the beam is turned on and is irradiating the target 302. That is, the radiation treatment beam 201 is not gated.

To summarize, in embodiments according to the invention, target motion is imaged in real time during treatment while the radiation treatment beam is on and is irradiating the target, and the angle and/or range of the beam and/or the position of the target are adapted in real time (while the radiation treatment beam is on and is irradiating the target) based on the acquired images.

FIG. 6 is a flowchart 600 of an example of computer-implemented operations for image-guided radiation therapy in embodiments according to the present invention. The flowchart 600 can be implemented by the radiation therapy system 200 using computer-executable instructions residing on some form of computer-readable storage medium (e.g., the control system 210 of FIG. 2). FIG. 6 is described with reference also to FIGS. 2 and 3.

In block 602 of FIG. 6, the radiation therapy system 200 acquires first images by directing a first imaging beam 331 from a first imaging beam source 321 through a target 302 and to a first imager 311.

In block 604, the radiation therapy system 200 acquires second images by directing a second imaging beam 332 from a second imaging beam source 322 through the target 302 and to the second imager 312.

In embodiments, the radiation therapy system 200 can filter the imaging beams as described above. In embodiments, the radiation therapy system 200 can select the voltage level at which the imaging beams 331 and 332 are produced so that the imaging beams have the same or different energies. In embodiments, the first imaging beam 331 and the second imaging beam 332 are each produced alternately at different voltages so that the imaging beams alternate between different energies.

In block 606, the radiation therapy system 200 generates a radiation treatment beam 201 and aims it at the target 302. While the operations of blocks 602, 604, and 606 are presented sequentially in this discussion, those operations can be performed in parallel.

In block 608, in response to movement of the target 302 and while the radiation treatment beam 201 is on and irradiating the target, the radiation therapy system 200 adjusts the radiation treatment beam and/or the position of the target to compensate for the movement and cause the beam to continue to intersect the target. That is, the radiation therapy system 200 can change the direction of the radiation treatment beam 201 and/or change the position of the target 302 to cause the beam to intersect the target and/or the energy of the beam to change the range of the beam, depending on the type (direction) of movement of the target, as described above.

FIG. 7 is a flowchart 700 of an example of computer-implemented operations for image-guided radiation therapy in embodiments according to the present invention. The flowchart 700 can be implemented as computer-executable instructions (e.g., the image-guided radiation therapy model 150 of FIG. 1) residing on some form of computer-readable storage medium (e.g., using the computing system 100 of FIG. 1). FIG. 7 is described with reference also to FIG. 3.

In block 702 of FIG. 7, first images are received by the control system 210 (controller) from a first imager 311. The first images show positions of a target 302. The first images were acquired by directing a first imaging beam 331 from a first imaging beam source 321 through the target 302 and to the first imager 311.

In block 704, second images are received by the control system 210 from a second imager 321. The second images show positions of the target 302. The second images were acquired by directing a second imaging beam 332 from a second imaging beam source 322 through the target 302 and to the second imager 312.

In block 706, the control system 210 detects a change in position of the target 302 based on the first images and the second images.

In block 708, in response to the change in position of the target 302, and while a radiation treatment beam 201 remains on and is irradiating the target, the control system 210 changes the radiation treatment beam and/or the position of the target to compensate for the change in position and cause the beam to continue to intersect the target. That is, the control system 210 can change the direction of the radiation treatment beam 201 and/or change the position of the target 302 to cause the beam to intersect the target and/or the energy of the beam to change the range of the beam, depending on the type (direction) of movement of the target, as described above.

In summary, embodiments according to the invention are able to accurately localize a target and surrounding healthy tissue. Different techniques, such as properly selected filters and imaging beam energies, can be used to make the target more visible. Consequently, for example, the radiation treatment beam can be interactively guided to provide a desired or optimal dose distribution into and across the target while sparing the surrounding healthy tissue. Breath control is therefore not required, making it more convenient for the patient and reducing the possibility of mistreatment due to improper breath control. Gating of the radiation treatment beam is also not required, thereby reducing treatment time and making treatment more convenient for the patient. Because the location of the target is known in real time, margins in the treatment plan (e.g., the planned irradiation volume) can be reduced without risk of compromising target dose coverage, lowering the risk of side effects.

In addition to IMRT and IMPT, embodiments according to the invention can be used in spatially fractionated radiation therapy including high-dose spatially fractionated grid radiation therapy and microbeam radiation therapy.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A system, comprising:
a first source of a radiation treatment beam;
a plurality of imagers comprising a first imager and a second imager;
a plurality of second sources comprising a first imaging beam source that directs a first imaging beam through a target and to the first imager to produce first images, and a second imaging beam source that directs a second imaging beam through the target and to the second imager to produce second images; and
a controller coupled to the first source, to the first and second imaging beam sources, and to the first and second imagers, wherein the controller uses the first images and the second images to detect a change in position of the target and, in response to the change, adjusts at least one of the radiation treatment beam and the position of the target to compensate for the change while the radiation treatment beam is on.

2. The system of claim 1, wherein the first imaging beam is produced at a first voltage and the second imaging beam is produced at a second voltage that is different from the first voltage.

3. The system of claim 1, wherein the first imaging beam is produced alternately at a first voltage and at a second voltage that is different from the first voltage, and wherein the second imaging beam is produced alternately at the first voltage and the second voltage.

4. The system of claim 1, further comprising a first filter positioned between the first imaging beam source and the first imager to filter the first imaging beam, and wherein a second filter positioned between the second imaging beam source and the second imager to filter the second imaging beam.

5. The system of claim 4, wherein the first filter comprises different regions comprising different filter materials, and wherein the second filter comprises different regions comprising different filter materials.

6. The system of claim 1, wherein the change comprises lateral movement of the target orthogonal to a direction of the radiation treatment beam, wherein the controller causes the radiation treatment beam to intersect the target by aiming the radiation treatment beam to follow movement of the target while the radiation treatment beam is on.

7. The system of claim 1, wherein the change comprises lateral movement of the target orthogonal to a direction of the radiation treatment beam, wherein the controller causes the radiation treatment beam to intersect the target by additional movement of the target while the radiation treatment beam is on.

8. The system of claim 1, wherein the change comprises movement of the target along a longitudinal axis of the radiation treatment beam, wherein the controller adjusts the energy of the radiation treatment beam to change the range of the radiation treatment beam to follow the movement of the target while the radiation treatment beam is on.

9. A method, comprising:
acquiring first images by directing a first imaging beam from a first imaging beam source through a target and to a first imager;
acquiring second images by directing a second imaging beam from a second imaging beam source through the target and to a second imager;
generating a radiation treatment beam; and
in response to movement of the target while the radiation treatment beam is on, adjusting at least one of the radiation treatment beam and a position of the target to compensate for the movement while the radiation treatment beam is on and irradiating the target, wherein the movement of the target and the position of the target is determined using the first images and the second images.

10. The method of claim 9, further comprising:
generating the first imaging beam at a first voltage; and
generating the second imaging beam at a second voltage that is different from the first voltage.

11. The method of claim 9, further comprising:
generating the first imaging beam alternately at a first voltage and at a second voltage that is different from the first voltage; and
generating the second imaging beam alternately at the first voltage and the second voltage.

12. The method of claim 9, further comprising:
filtering the first imaging beam with a first filter before the first imaging beam reaches the first imager; and
filtering the second imaging beam with a second filter before the second imaging beam reaches the second imager.

13. The method of claim 12, wherein the first filter comprises different regions comprising different filter materials, and wherein the second filter comprises different regions comprising different filter materials.

14. The method of claim 9, wherein said adjusting comprises an operation selected from the group consisting of: changing a direction of the radiation treatment beam; changing a position of the target to cause the radiation treatment beam to intersect the target; and adjusting the energy of the radiation treatment beam to change the range of the radiation treatment beam.

15. A method, comprising:
receiving first images from a first imager, wherein the first images were acquired by directing a first imaging beam from a first imaging beam source through a target and to the first imager;
receiving second images from a second imager, wherein the second images were acquired by directing a second imaging beam from a second imaging beam source through the target and to the second imager;
directing a radiation treatment beam at the target and, while the radiation treatment beam is on, detecting a change in position of the target based on the first images and the second images; and
in response to the change in position of the target, while the radiation treatment beam is on, changing at least one of the radiation treatment beam and the position of the target to cause the radiation treatment beam to intersect the target.

16. The method of claim 15, wherein the first imaging beam is produced at a first voltage and the second imaging beam is produced at a second voltage that is different from the first voltage.

17. The method of claim 15, wherein the first imaging beam is produced alternately at a first voltage and at a second voltage that is different from the first voltage, and wherein the second imaging beam is produced alternately at the first voltage and at the second voltage.

18. The method of claim 15, wherein the first imaging beam is filtered with a first filter before the first imaging beam reaches the first imager, and wherein the second imaging beam is filtered with a second filter before the second imaging beam reaches the second imager.

19. The method of claim 18, wherein the first filter comprises different regions comprising different filter materials, and wherein the second filter comprises different regions comprising different filter materials.

20. The method of claim 15, wherein said changing comprises an operation selected from the group consisting of: changing a direction of the radiation treatment beam; changing the position of the target to cause the radiation treatment beam to intersect the target; and adjusting the energy of the radiation treatment beam to change the range of the radiation treatment beam.
